(19) United States
(12) Patent Application Publication
(10) Pub. No.: US 2013/0158363 A1
(43) Pub. Date: Jun. 20, 2013

(54) INTEGRATED, HAND-HELD APPARATUS AND ASSOCIATED METHOD FOR ACQUIRING DIAGNOSTIC AND PROGNOSTIC INFORMATION FROM A PATIENT AT THE BEDSIDE OR AT SOME OTHER PATIENT LOCATION

(71) Applicant: William Zoghbi, Houston, TX (US)

(72) Inventor: William Zoghbi, Houston, TX (US)

(21) Appl. No.: 13/702,424

(22) PCT Filed: Jun. 13, 2011
(86) PCT No.: PCT/US11/40209

§ 371 Date: Feb. 20, 2013

Related U.S. Application Data
(60) Provisional application No. 61/353,859, filed on Jun. 11, 2010.

(51) Int. Cl. A61B 8/00; A61B 8/14; A61B 5/00; A61B 8/08; A61B 5/04; A61B 5/044; A61B 5/1455; A61B 5/01; A61B 5/0205; A61B 7/00; A61B 5/0408

(52) U.S. Cl. CPC A61B 8/4416; A61B 7/00; A61B 8/14; A61B 8/461; A61B 5/7405; A61B 8/565; A61B 5/7246; A61B 8/52; A61B 5/04085; A61B 5/044; A61B 5/14551; A61B 5/01; A61B 5/02055; A61B 5/0006; A61B 5/0008; A61B 5/0013; A61B 5/04012; A61B 5/4836
USPC 600/301

(57) ABSTRACT

An integrated, hand-held apparatus for acquiring diagnostic and prognostic information from a patient at the bedside or at some other patient location, the apparatus including a wand with a microphone for acquiring sound information from the patient and an ultrasound emitter/receiver for acquiring image data from the patient, a base unit including a speaker for presenting sound information to a user and a display for presenting image information to a user, and transferring the sound information acquired by the microphone and the image information acquired by the ultrasound emitter/receiver from the wand to the base unit.

### REFERENCE TO PENDING PRIOR PATENT APPLICATION

This patent application claims benefit of pending prior U.S. Provisional Patent Application Ser. No. 61/353,859, filed Jun. 11, 2010 by William Zoghbi for OMNISCOPE (Attorney's Docket No. 058001.105026; ZOGHBI-1 PROV), which patent application is hereby incorporated herein by reference.

### FIELD OF THE INVENTION

This invention relates to medical apparatus and methods in general, and more particularly to medical apparatus and methods for acquiring diagnostic and prognostic information from a patient at the bedside or at some other patient location.

### BACKGROUND OF THE INVENTION

A healthcare professional (e.g., doctor, nurse, etc.) must frequently acquire diagnostic and prognostic information from a patient “at the bedside” or at some other patient location. In some cases, this diagnostic and prognostic information may be acquired by interviewing the patient (or an accompanying individual), e.g., by asking the patient's age, medical history, etc. And in some cases this diagnostic and prognostic information may be acquired by the visual and tactile examination of the patient, e.g., by observing the appearance of the patient, sensing tissue tone and texture, etc. And in some cases this diagnostic and prognostic information may be acquired through the use of medical apparatus, e.g., a stethoscope may be used to acquire sounds from the patient's body (heart, vasculature, lungs, abdomen, etc.), an ultrasound machine may be used to acquire images of internal anatomy (organs, vasculature, etc.), an electrocardiogram (ECG) machine may be used to acquire electrical signals from the patient's body, etc.

The present invention is directed to situations where a medical apparatus is used to acquire diagnostic and prognostic information from the patient at the bedside or at some other patient location.

Current apparatus for acquiring diagnostic and prognostic information from the patient at the bedside or at some other patient location tend to suffer from a number of deficiencies.

By way of example but not limitation, stethoscopes are commonly utilized to acquire real-time sound information from the patient. However, this real-time sound information is subject to the immediate, on-the-fly, individualized interpretation of the healthcare professional who is using the stethoscope and is not stored for subsequent evaluation by that same healthcare professional or by others (or for comparison against a database of sound information from that patient or from other patients, either by that healthcare professional or by other healthcare professionals or by a computerized “comparison engine” or “auto-interpretation engine” which can compare the current sound information against a library of sound information and its anatomical meaning so as to provide further assistance to the healthcare professional). Unfortunately, current stethoscopic examination is a highly subjective process, dependent on the specific listening abilities and interpretive skills of the individual healthcare professional, and it has been estimated that the average healthcare professional has less than a 30% chance of properly diagnosing heart murmurs by stethoscopic examination.

By way of further example but not limitation, ultrasound machines are typically relatively large, cart-mounted devices which must be pre-positioned at the patient's bedside (or at some other patient location) or moved to the patient's bedside (or to some other patient location) prior to use. One portable hand-held ultrasound-only device has recently become available in the marketplace, but this device is not normally carried by most healthcare professionals as part of their routine equipment due to cost and convenience considerations. Due to these logistical limitations, ultrasound machines are typically not used as frequently as would be desirable to acquire diagnostic and prognostic information from the patient at the bedside or at some other patient location.

By way of still further example but not limitation, ECG machines tend to share the same size and logistical limitations as ultrasound machines, i.e., even where they are made smaller or portable, they are still an ECG-only device which is not normally carried by most healthcare professionals as part of their routine equipment due to cost and convenience considerations. Hence ECG machines are also not used as frequently as would be desirable to acquire diagnostic and prognostic information from the patient at the bedside or at some other patient location. Furthermore, the standard 12-lead ECG machine generally takes some time and skill to set up, further limiting its use at the bedside or at some other patient location.

Significantly, each of the foregoing devices (i.e., stethoscope, ultrasound machine and ECG machine) is currently a separate, self-standing piece of equipment. Accordingly, in order to obtain the diagnostic and prognostic information available from each of these devices, multiple pieces of equipment must be used by the healthcare professional in order to acquire a full set of patient data. However, due to cost, convenience and logistical issues, the healthcare professional often does not have all three of these devices readily available at the patient's bedside or at some other patient location, and hence the healthcare professional is often required to make a diagnosis without the benefit of all of the diagnostic and prognostic information that would be available if all three devices were present at the patient's bedside in a single, convenient, portable device.

By way of example but not limitation, having sound information (auscultation) and image data (ultrasound) simultaneously available to the healthcare professional at the bedside or some other patient location would be extremely useful in properly diagnosing and identifying fluid build-ups in the body (e.g., in the chest, limbs or abdomen) such as by palpation in conjunction with sound information and/or image data, determining appropriate biopsy sites and obtaining desired biopsy specimens, identifying and accessing (e.g., via instrument guidance) desired interventional sites, accurately locating blood vessels, etc. Furthermore, the use of sound information (auscultation) with Doppler ultrasound could lead to better specificity and better diagnosis of a patient condition than sound information (auscultation) alone (e.g., immediate diagnosis of murmurs or bruits).

Furthermore, having two or more diagnostic and prognostic functions (e.g., sound information and image data) simultaneously available to the healthcare professional at the bedside or some other patient location could provide additional significant advantages. By way of example but not limitation, the following advantages could be obtained:
(i) accuracy advantage—by having two or more functions immediately at hand (instead of available as the result of performing two or more tests separated over time), the healthcare professional could be better able to localize a problem (e.g., the healthcare professional could hear an issue via auscultation, and could then immediately run an ultrasound to visualize the problem, instead of having to wait for the results of a separate ultrasound test performed by a different healthcare professional at a later time);
(ii) diagnostic advantage—by having two or more functions immediately at hand (instead of available as the result of performing two or more tests separated over time), the healthcare professional could be better able to isolate/detect a problem while the patient is actually experiencing the problem (e.g., the healthcare professional could hear, see and correlate the problem with other key biological measures while the patient is experiencing and describing the symptoms, instead of having to wait for the results of later-scheduled tests);
(iii) prognostic advantage—by having two or more functions immediately at hand (instead of available as the result of performing two or more tests separated over time), the healthcare professional could be better able to gauge the severity of a problem (e.g., the healthcare professional could hear, see and correlate the problem with other key biological measures contemporaneously, as opposed to having to wait for the results of tests separated over time);
(iv) economic advantage—by having two or more functions immediately at hand (instead of available as the result of performing two or more tests separated over time), it may be possible to avoid multiple patient trips to a healthcare facility and to avoid involving multiple healthcare professionals in the process, thereby reducing patient inconvenience and patient opportunity cost, reducing administrative costs, and increasing the productivity of the healthcare professionals;
(v) treatment advantage—by having two or more functions immediately at hand (instead of available as the result of performing two or more tests separated over time), there could be a reduction of the time between tests, which could permit treatment to be more rapidly administered to the patient and could potentially avoid a worsening of the patient's condition;
(vi) convenience advantage—by having two or more functions immediately at hand (instead of available as the result of two or more tests separated over time), there could be a reduction in the number of appointments which need to be scheduled by/for the patient and there could be a reduction in the number of immediate follow-up appointments which need to be scheduled by/for the patient;
(vii) health advantage—by having two or more functions immediately at hand (instead of available as the result of two or more tests separated over time), there could be a reduction of the time during which the patient may be anxious about an undiagnosed condition, thereby reducing overall patient anxiety and potentially improving patient health as a result (e.g., there could be a reduction of the time during which the patient is waiting for an appointment for a test to be conducted, for the results to be acquired by a technician, for the results to be provided by the technician to the doctor, for the results to be interpreted by the doctor, and for the results to be provided by the doctor to the patient, during which time the patient may be suffering through the anxiety of having an undiagnosed condition).

Thus there is a need for a new and improved apparatus for acquiring diagnostic and prognostic information from a patient at the bedside or at some other patient location which does not suffer from the aforementioned deficiencies of the prior art.

### SUMMARY OF THE INVENTION

These and other objects of the present invention are addressed by the provision and use of novel apparatus for acquiring diagnostic and prognostic information from a patient at the bedside or at some other patient location. More particularly, in accordance with the present invention, there is provided a novel apparatus for acquiring diagnostic and prognostic information from a patient at the bedside or at some other patient location, wherein the novel apparatus comprises an integrated, hand-held device which is intended to be conveniently carried by the healthcare professional on their person (e.g., in the manner of a conventional stethoscope) and which can be used to acquire sound, image and preferably also electrical and other (e.g., patient history, blood pressure, blood oximetry, patient temperature, etc.) information from the patient, so as to enable the healthcare professional to carry out a rapid, accurate and comprehensive objective physical examination of the patient at the bedside or some other patient location, including cardiovascular diagnostics and prognostics, regardless of any other equipment that may be available at that location, and/or to aid/guide the healthcare professional in therapeutic interventions (e.g., localization of pericardial or pleural effusions and guiding a needle/catheter to drain the fluid), and to store the diagnostic and prognostic information acquired from the patient, either locally on the device or externally on an external network, for later review by that same healthcare professional and/or by others. Significantly, because the novel integrated, hand-held apparatus of the present invention facilitates the acquisition of objective diagnostic and prognostic information from the patient, the present invention facilitates a more accurate and prompt diagnosis and prognosis of patient conditions by the clinician and also allows a broader set of healthcare professionals (e.g., technicians and others who will not actually render a diagnosis) to be involved in the acquisition of diagnostic and prognostic information from the patient.

In one form of the present invention, there is provided an integrated, hand-held apparatus for acquiring diagnostic and prognostic information from a patient at the bedside or at some other patient location, the apparatus comprising:

a wand comprising:
a microphone for acquiring sound information from the patient; and
an ultrasound emitter/receiver for acquiring image data from the patient; and

a base unit comprising:
a speaker for presenting sound information to a user; and
a display for presenting image information to a user; and

transferring means for transferring the sound information acquired by the microphone, and the image information acquired by the ultrasound emitter/receiver, from the wand to the base unit.

In another form of the present invention, there is provided a wand comprising:
a microphone for acquiring sound information from the patient; and
an ultrasound emitter/receiver for acquiring image data from the patient.

In another form of the present invention, there is provided an integrated, hand-held apparatus for acquiring diagnostic and prognostic information from a patient at the bedside or at some other patient location, the apparatus comprising:

a wand comprising:
a microphone for acquiring sound information from the patient; and
an ultrasound emitter/receiver for acquiring image data from the patient;

a base unit comprising:
a speaker for presenting sound information to a user; and
a display for presenting image information to a user;

transferring means for transferring the sound information acquired by the microphone, and the image information acquired by the ultrasound emitter/receiver, from the wand to the base unit;

a plurality of electrodes for acquiring electrical information from the patient, and means for displaying the electrical information acquired from the patient on at least one of the display and the speaker;

a blood pressure cuff for acquiring blood pressure information from the patient, means for transferring the blood pressure information from the blood pressure cuff to the base unit, and means for displaying blood pressure information on at least one of the display and the speaker;

a pulse oximeter for acquiring pulse rate and SpO₂ information from the patient, means for transferring the pulse rate and SpO₂ information from the blood pulse oximeter to the base unit, and means for displaying pulse rate and SpO₂ information on at least one of the display and the speaker;

a temperature sensor for acquiring temperature information from the patient, means for transferring the temperature information from the temperature sensor to the base unit, and means for displaying temperature information on at least one of the display and the speaker; and

communication means for permitting the apparatus to communicate with an external network.

In another form of the present invention, there is provided a method for acquiring diagnostic and prognostic information from a patient at the bedside or at some other patient location, the method comprising:

providing an integrated, hand-held apparatus comprising:
a wand comprising:
   a microphone for acquiring sound information from the patient; and
   an ultrasound emitter/receiver for acquiring image data from the patient; and
a base unit comprising:
   a speaker for presenting sound information to a user; and
   a display for presenting image information to a user; and
transferring means for transferring the sound information acquired by the microphone, and the image information acquired by the ultrasound emitter/receiver, from the wand to the base unit; and

acquiring diagnostic and prognostic information from the patient using the apparatus.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view showing the front side of a novel integrated, hand-held apparatus for acquiring diagnostic and prognostic information from a patient at the bedside or at some other patient location, wherein the novel apparatus comprises a base unit and a wand;

FIG. 2 is a schematic view showing the rear side of the novel integrated, hand-held apparatus shown in FIG. 1;

FIG. 3 is a schematic view showing the internal system components of the base unit of the novel integrated, hand-held apparatus shown in FIG. 1;

FIG. 4 is a schematic view showing the internal system components of the wand of the novel integrated, hand-held apparatus shown in FIG. 1;

FIGS. 4A-4C are schematic views showing additional configurations for the novel integrated, hand-held apparatus shown in FIG. 1;

FIG. 5 is a schematic view showing the novel integrated, hand-held apparatus of FIG. 1 being used to acquire sound information from the body of a patient;

FIGS. 6 and 7 are schematic views showing the novel integrated, hand-held apparatus of FIG. 1 being used to acquire image information from the body of a patient;

FIG. 8 is a schematic view showing integration of the novel integrated, hand-held apparatus of FIG. 1 with an external network;

FIGS. 9 and 10 show alternative constructions for the wand of the novel integrated, hand-held apparatus shown in FIG. 1;

FIG. 11 is a schematic view showing the novel integrated, hand-held apparatus of FIG. 1 being used to acquire electrical information from the body of a patient;

FIG. 12 is a schematic view showing a novel pressure cuff which may be used with the novel integrated, hand-held apparatus of FIG. 1 to acquire blood pressure information from the body of a patient;

FIG. 13 is a schematic view showing a novel pulse oximeter which may be used with the novel integrated, hand-held apparatus of FIG. 1 to acquire pulse rate information and SpO₂ information from the body of a patient;

FIG. 14 is a schematic view showing a novel temperature monitor which may be used with the novel integrated, hand-held apparatus of FIG. 1 to acquire temperature information from the body of a patient;

FIG. 15 is a schematic view showing how a docking station may be used with the novel integrated, hand-held apparatus of FIG. 1, and also showing further aspects of the present invention;

FIG. 16 is a schematic view showing one exemplary architecture which may be employed in the construction of the novel integrated, hand-held apparatus of FIG. 1;

FIGS. 17-33 are schematic views showing alternative constructions for the wand of the novel integrated, hand-held apparatus of the present invention;

FIG. 34 is a schematic view showing an alternative construction for a novel integrated, hand-held apparatus for acquiring diagnostic and prognostic information from a patient at the bedside or at some other patient location, wherein the novel apparatus comprises a “unibody” construction;

FIGS. 35-37 are schematic views showing one approach for the “unibody” construction of FIG. 34; and

FIGS. 38 and 39 are schematic views showing another approach for the “unibody” construction of FIG. 34.

### DETAILED DESCRIPTION OF THE PREFERRED EMBODIMENTS

### Basic Device

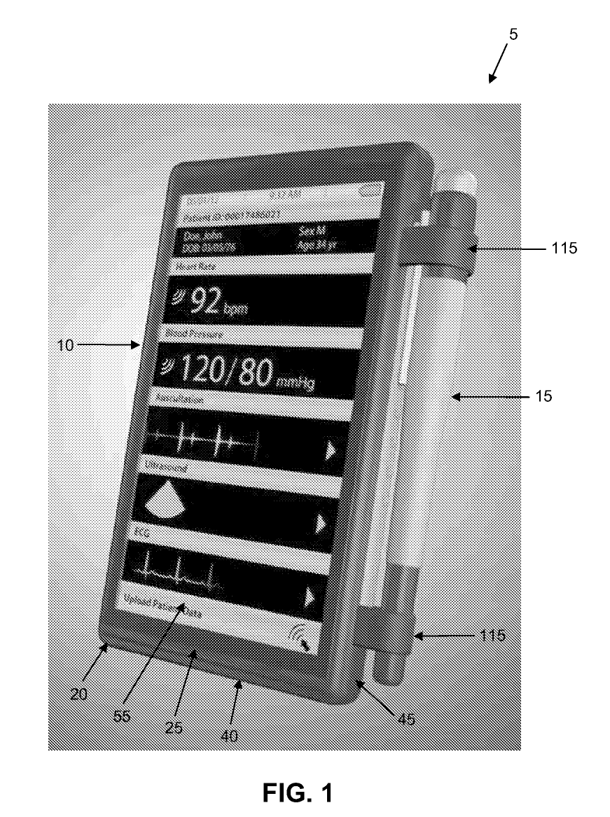

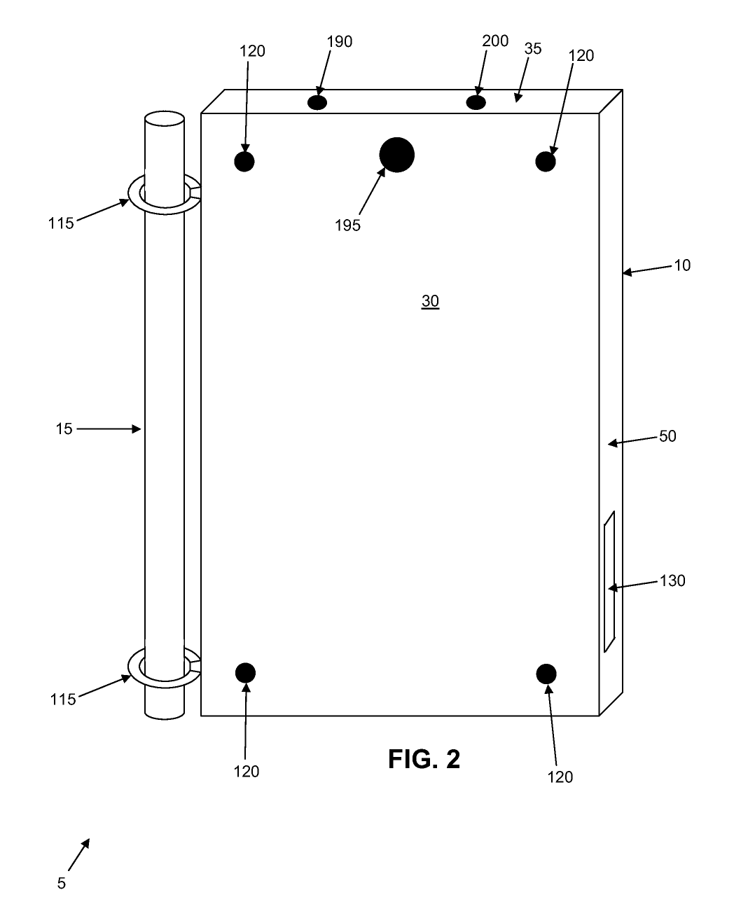

Looking first at FIGS. 1 and 2, there is shown novel apparatus 5 for acquiring diagnostic and prognostic information from a patient at the bedside or at some other patient location. Novel apparatus 5 comprises an integrated, hand-held device which is intended to be conveniently carried by a healthcare professional on their person (e.g., in the manner of a conventional stethoscope) and which can be used to acquire sound, image and preferably also electrical and other (e.g., patient history, blood pressure, blood oximetry, patient temperature, etc.) information from the patient, so as to enable the healthcare professional to carry out a rapid, accurate and comprehensive objective physical examination of the patient at the bedside or at some other patient location, including cardiovascular diagnostics and prognostics, regardless of any other equipment that may be available at that location, and/or to aid/guide the healthcare professional in therapeutic interventions (e.g., localization of pericardial or pleural effusions and guiding a needle/catheter to drain the fluid), and to store the diagnostic and prognostic information acquired from the patient, either locally on the device or externally on an external network, for later review by that same healthcare professional and/or by others. Significantly, because the novel integrated, hand-held apparatus of the present invention facilitates the acquisition of objective diagnostic and prognostic information from the patient, the present invention facilitates a more accurate and prompt diagnosis and prognosis of patient conditions by the clinician and also allows a broader set of individuals (e.g., technicians and others who will not actually render the diagnosis) to be involved in the acquisition of diagnostic and prognostic information from the patient.

Novel integrated, hand-held apparatus 5 generally comprises a base unit 10 and a wand 15 which is preferably releasably mounted to the base unit.

Base unit 10 comprises a body 20 generally characterized by a front side 25, a rear side 30, a top end 35, a bottom end 40, a right side 45 and a left side 50. A touchscreen display 55 is mounted to front side 25 of body 20. Body 20 of base unit 10 is sized so as to be conveniently hand-held by a healthcare professional.

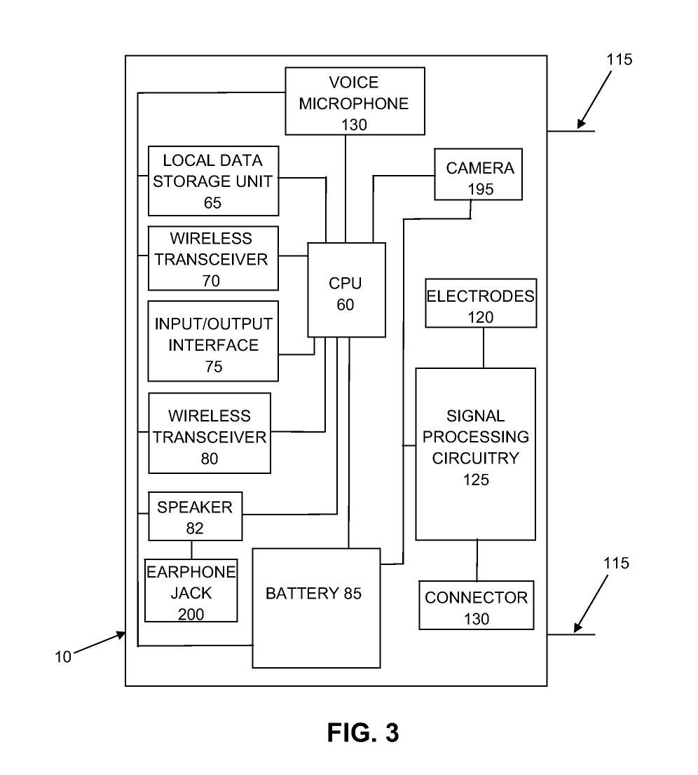

As seen in FIG. 3, the interior of base unit 10 comprises various electrical components for providing novel apparatus 5 with its functional capabilities. More particularly, in one preferred embodiment, novel apparatus 5 generally comprises a central processing unit (CPU) 60, a local data storage unit 65 (e.g., non-volatile semiconductor memory), a wireless transceiver 70 (e.g., a Bluetooth device, etc.) for communicating with wand 15 (and/or with other apparatus, as will hereinafter be discussed), an input/output interface 75 for driving touchscreen display 55, a wireless transceiver 80 (e.g., a WiFi device) for communicating with an external network (e.g., a wireless computer network operated by a healthcare facility such as a hospital), a speaker 82 for presenting sound information to the healthcare professional, and a power source (e.g., battery) 85 for powering the aforementioned electrical components, as well as any other electrical components that might be provided in base unit 10.

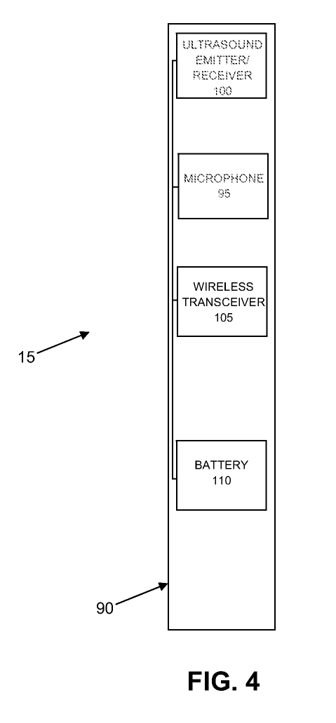

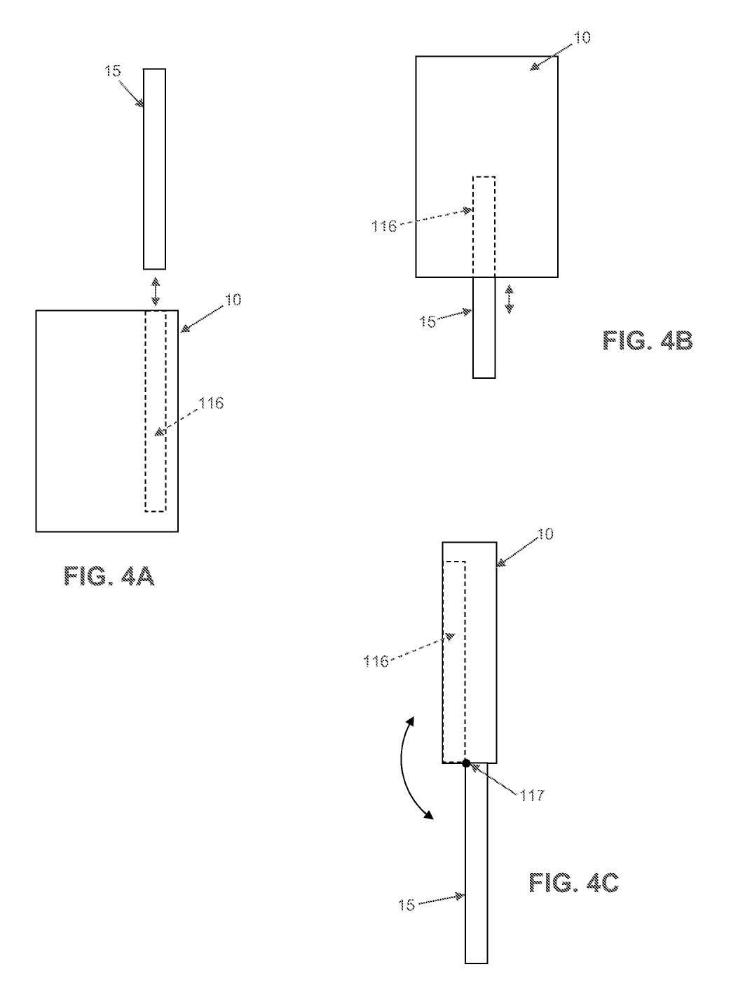

Looking next at FIG. 4, wand 15 comprises a body 90 for carrying the various electrical components which provide the wand with its functional capabilities. Body 90 of wand 15 is sized to be conveniently hand-held by a healthcare professional. More particularly, in one preferred form of the invention, wand 15 has an elongated, generally cylindrical body 90, and wand 15 comprises a microphone 95 for acquiring sound information from the patient, an ultrasound emitter/receiver 100 for acquiring image data from the patient, a wireless transceiver 105 (e.g., a Bluetooth device) for communicating with wireless transceiver 70 of base unit 10, and a power source (e.g., battery) 110 for powering the aforementioned electrical components of wand 15, as well as any other electrical components that might be provided on wand 15. In this respect it should be appreciated that the ultrasound emitter/receiver 100 used to acquire image data from the patient may be of the sort well known in the art of ultrasound imaging, e.g., it may comprise piezoelectric crystals configured for ultrasound operation using various modes (including phased array technology) in the 2 MHz-7 MHz range. Ultrasound emitter/receiver 100 is preferably also configured to perform Doppler ultrasound (color Doppler and spectral), e.g., for analyzing blood flow.

Preferably base unit 10 includes one or more retainers 115 (FIGS. 1-3) for releasably holding wand 15 to base unit 10. Alternatively, base unit 10 may include a recess for receiving wand 15 therein, so that wand 15 may be stored within base unit 10 while the wand is not in use (thereby providing a more integrated form factor which may facilitate insertion/removal from a pocket of the healthcare professional and/or provide other convenience in carrying, storage, etc.), with the wand being fully or partially removable from the recess when the wand is to be used. See, for example, FIG. 4A, where wand 15 may be releasably stored in a recess 116 formed in base unit 10; FIG. 4B, where wand 15 may selectively project out of a recess 116 formed in base unit 10; and FIG. 4C, where wand 15 may be hinged to base unit 10 at a hinge 117 such that wand 15 may fold into a recess 116 formed in base unit 10.

In accordance with the present invention, the healthcare professional is intended to carry novel integrated, hand-held apparatus 5 on their person as they move throughout a healthcare facility (e.g., a hospital). When the healthcare professional wishes to acquire diagnostic and prognostic information from a patient at the bedside or at some other patient location, the healthcare professional may use touchscreen display 55 (FIG. 1) to navigate between the different functional capabilities of novel apparatus 5.

By way of example but not limitation, the healthcare professional may use touchscreen display 55 to store text information about the patient (e.g., text information acquired by interviewing the patient or an accompanying individual). This information may be stored locally on base unit 10 in local data storage unit 65 and/or uploaded to an external network via wireless transceiver 80 in base unit 10 (e.g., to be added to an Electronic Health Record (EHR)).

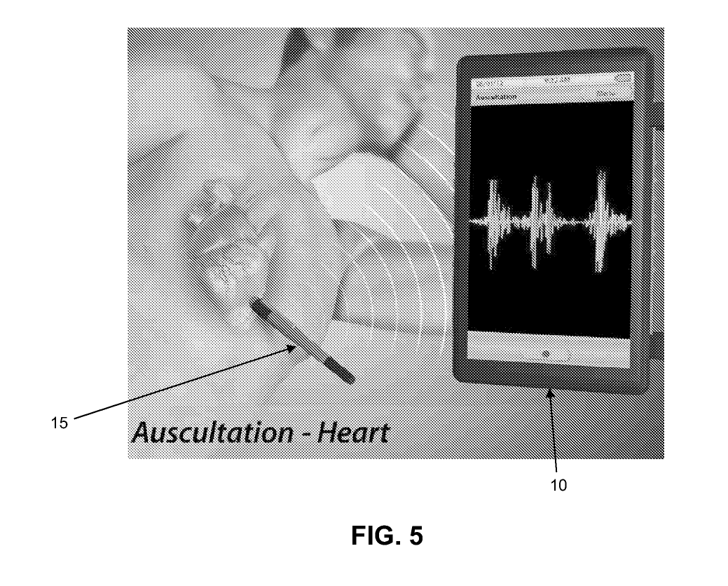

The healthcare professional may also use microphone 95 on wand 15 to acquire sound information from the patient, e.g., sounds from the heart, vasculature, lungs, abdomen, etc. See FIG. 5. The sound information acquired by microphone 95 on wand 15 is transmitted to base unit 10 via wireless transceiver 105 on wand 15 and wireless transceiver 70 on base unit 10. Base unit 10 may then present this sound information to the healthcare professional, e.g., audibly via speaker 82 and/or visually via touchscreen display 55. Preferably this sound information is also simultaneously stored on base unit 10 in local data storage unit 65 and/or uploaded to an external network via wireless transceiver 80 in base unit 10 (e.g., to be added to an Electronic Health Record (EHR)).

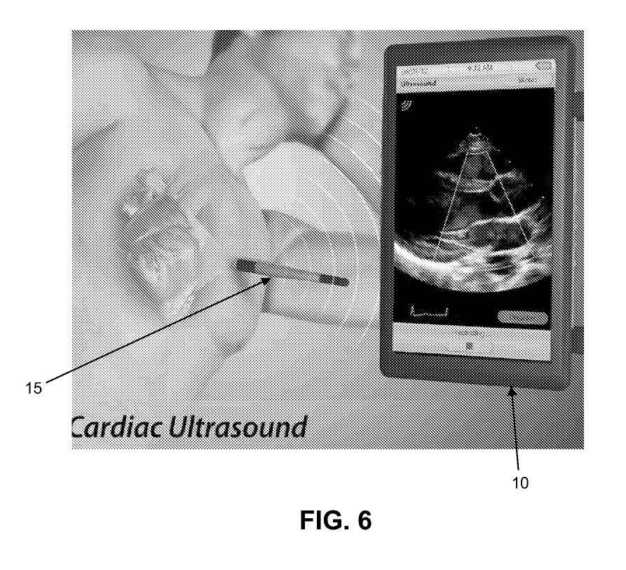

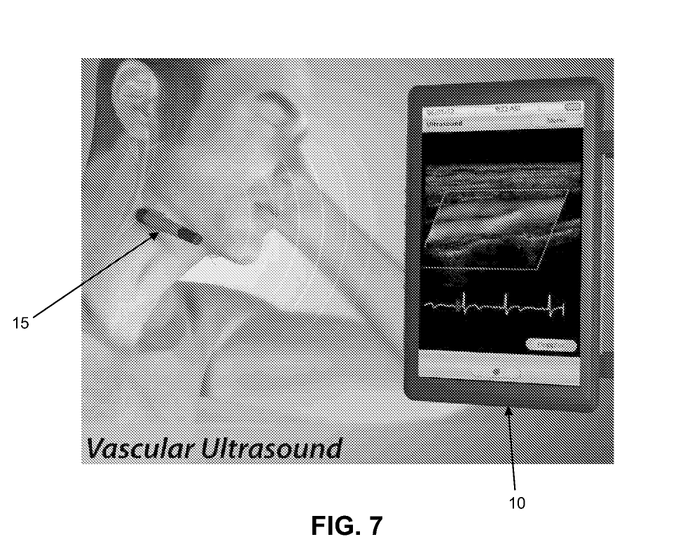

The healthcare professional may also use ultrasound emitter/receiver 100 on wand 15 to acquire image information from the patient, e.g., images of the heart, vasculature, lungs, abdomen, etc. See FIGS. 6 and 7. The image information acquired by ultrasound emitter/receiver 100 on wand 15 is transmitted to base unit 10 via wireless transceiver 105 on wand 15 and wireless transceiver 70 on base unit 10. Base unit 10 may then present this image information to the healthcare professional visually via touchscreen display 55. Preferably this image information is also simultaneously stored on base unit 10 in local data storage unit 65 and/or uploaded to an external network via wireless transceiver 80 in base unit 10 (e.g., to be added to an Electronic Health Record (EHR)).

The sound information acquired from the patient by novel apparatus 5, and/or the image data acquired from the patient by novel apparatus 5, may be used directly by the healthcare professional to make a diagnosis and prognosis. Additionally and/or alternatively, the sound information and/or image data may be used by a computerized “comparison engine” or “auto-interpretation” engine (which can be incorporated in novel integrated, hand-held apparatus 5) to provide additional diagnostic and prognostic information to the healthcare professional.

Additionally, the sound information acquired from the patient by novel apparatus 5, and/or the image data acquired from the patient by novel apparatus 5, may be used by the healthcare professional to aid/guide the healthcare professional in therapeutic interventions (e.g., localization of pericardial or pleural effusions and guiding a needle/catheter to drain the fluid).

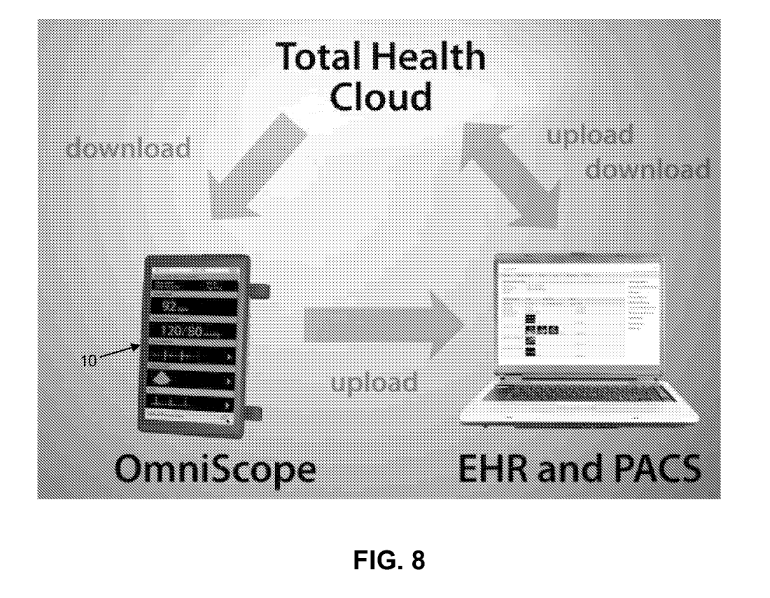

In addition to the foregoing, novel integrated, hand-held apparatus 5 may also acquire data (e.g., text, sounds, images, etc.) from an external network (e.g., a wireless computer network operated by a healthcare facility such as a hospital) via wireless transceiver 80 in base unit 10 (e.g., to access information from an Electronic Health Record (EHR)) and present that data to the healthcare professional via touchscreen display 55 and/or speaker 82. See FIG. 8. In this way, novel integrated, hand-held apparatus 5 also provides the healthcare professional with access to patient records which may be available from the external network, whereby to further assist the healthcare professional in the diagnosis of the patient. Significantly, such access to patient records allows the current diagnostic and prognostic information acquired by novel integrated, hand-held apparatus 5 to be compared with the historic diagnostic and prognostic information in the patient records, which can assist the healthcare professional in the diagnosis of the patient. This comparison of current vs. historic diagnostic and prognostic information can be effected by the healthcare professional themselves or by a computerized “comparison engine” or “auto-interpretation engine” which can be incorporated in novel integrated, hand-held apparatus 5 or provided by the external network. In this respect it will be appreciated that the “auto-interpretation engine” can compare the current diagnostic and prognostic information against a library of diagnostic and prognostic information and its anatomical meaning (e.g., heart murmur, valve issues, etc.) so as to provide further assistance to the healthcare professional.

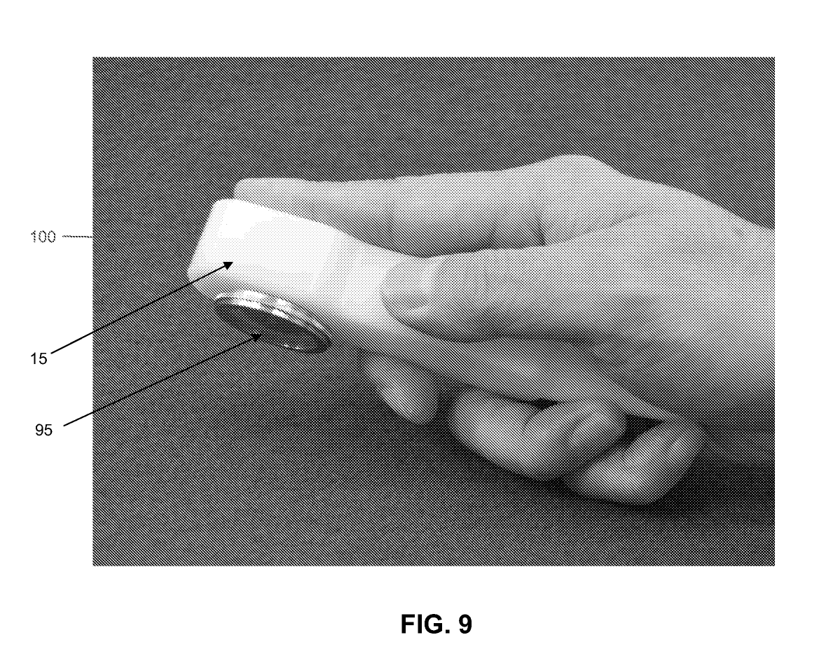

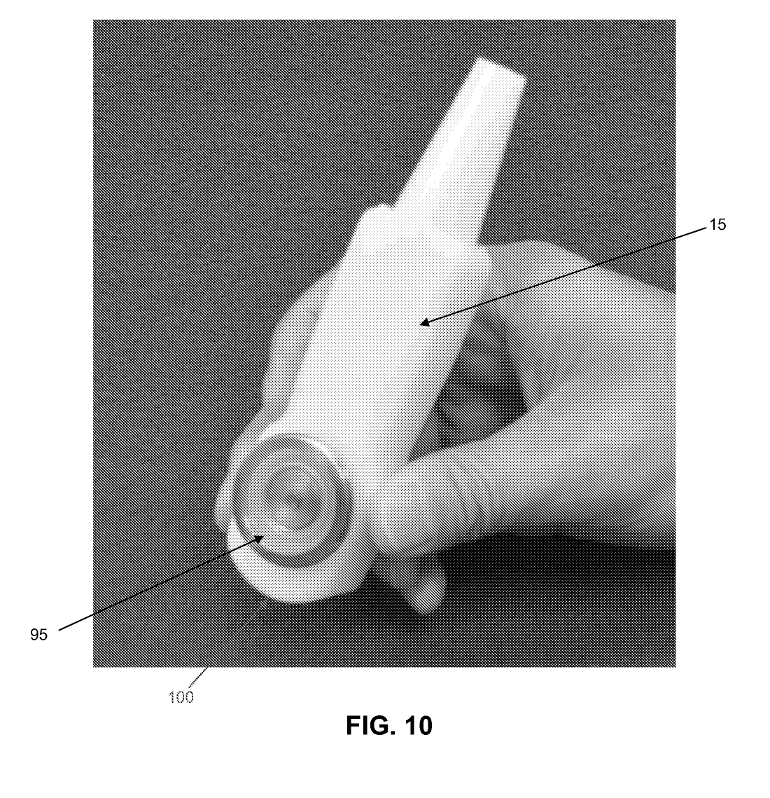

As noted above, wand 15 comprises a microphone 95 for acquiring sound information from the patient, and an ultrasound emitter/receiver 100 for acquiring image data from the patient. If desired, wand 15 may be constructed so that microphone 95 and ultrasound emitter/receiver 100 are both presented at an end of the elongated, generally cylindrical body 90 of wand 15. See, for example, FIGS. 5-7. In this case, if desired, microphone 95 and ultrasound emitter/receiver 100 may be disposed adjacent to one another at one end of wand 15 (e.g., longitudinally adjacent to one another or laterally adjacent to one another), and/or share common components. Or microphone 95 and ultrasound emitter/receiver 100 may be disposed at opposite ends of wand 15. Alternatively, if desired, wand 15 may be constructed so that one component (e.g., microphone 95) is presented intermediate the length of the wand 15 and the other component (e.g., ultrasound emitter/receiver 100) is presented at one end of the wand 15. In this case, it may be desirable to form body 90 of wand 15 with something other than a generally cylindrical configuration. See, for example, FIGS. 9 and 10, which show body 90 of wand 15 having a more complex configuration, and with microphone 95 intermediate the length of wand 15 and ultrasound emitter/receiver 100 at one end of the wand.

### ECG Electrodes

In one preferred form of the present invention, and looking now at FIG. 2, base unit 10 of novel integrated, hand-held apparatus 5 also comprises a plurality of electrodes 120 for acquiring electrical information from the patient. To this end, base unit 10 also comprises signal processing circuitry 125 (FIG. 3) for converting the analog electrical signals detected by electrodes 120 into the digital electrical signals required by CPU 60.

In one preferred form of the present invention, there are four electrodes 120, and these four electrodes 120 are disposed adjacent the four corners of rear side 30 of base unit 10, in the manner shown in FIG. 2. In another preferred form of the invention, there may be more or less than four electrodes 120, and the electrodes 120 are arranged in a predetermined pattern on the rear side 30 of base unit 10, with this predetermined pattern being configured so as to facilitate the optimal acquisition of selected electrical signals from the body of the patient (e.g., the predetermined pattern may be configured so as to optimize the acquisition of particular types of cardiac signals from the body of the patient). In this respect it should be appreciated that, in either case, it is not intended that electrodes 120 be configured in the specific electrode pattern used in a standard 12-lead ECG procedure; however, it should also be appreciated that important electrical information can be acquired from the patient's body without using a standard 12-lead ECG electrode array, and the plurality of electrodes 120 provided on base unit 10 allow the healthcare professional to acquire some of this important electrical information. Thus it will be understood that the electrical information acquired by the plurality of electrodes 120 can be highly useful to a healthcare professional diagnosing the patient even though electrodes 120 are not configured in a standard 12-lead ECG electrode array. By way of example but not limitation, the provision of electrodes 120 on novel apparatus 5 allows the healthcare professional to get a quick insight into any heart rhythm abnormalities of the patient almost immediately, without waiting for a traditional 12-lead ECG procedure to be performed.

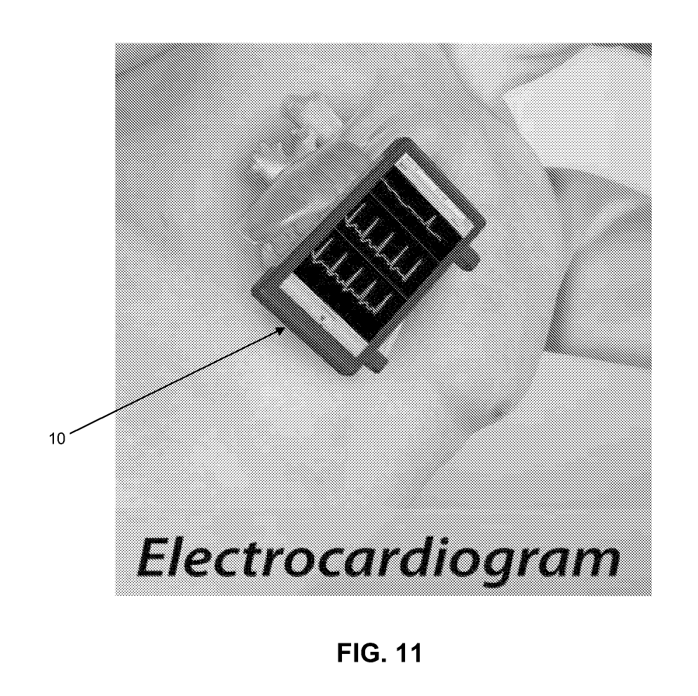

In accordance with the present invention, when the healthcare professional wishes to acquire electrical information from a patient at the bedside or at some other patient location, the healthcare professional positions base unit 10 of novel integrated, hand-held apparatus 10 against the skin of the patient, with electrodes 120 contacting the skin of the patient, so that electrical information is acquired from the body of the patient by electrodes 120. The electrical information acquired by electrodes 120 is processed by signal processing circuitry 125 and then passed to CPU 60. As a result, the electrical information acquired by electrodes 120 may then be presented to the healthcare professional, e.g., visually via touchscreen display 55 and/or audibly via speaker 82. See FIG. 11. Preferably this electrical information is also simultaneously stored locally on base unit 10 in local data storage unit 65 and/or uploaded to an external network (e.g., a wireless computer network operated by a healthcare facility such as a hospital) via wireless transceiver 80 in base unit 10 (e.g., to be added to an Electronic Health Record (EHR)).

Also in accordance with the present invention, base unit 10 preferably includes a connector 130 (FIGS. 2 and 3) for connecting a standard 12-lead ECG electrode array to base unit 10 of novel integrated, hand-held apparatus 5. As a result, once a standard 12-lead ECG electrode array is connected to base unit 10 via connector 130, the electrical information acquired by the standard 12-lead ECG electrode array may be processed by signal processing circuitry 125 and CPU 60, and then presented to the healthcare professional, e.g., visually via touchscreen display 55 and/or audibly via speaker 82. Preferably the electrical information acquired from the standard 12-lead ECG electrode array connected to connector 130 is also simultaneously stored locally on base unit 10 in local data storage unit 65 and/or uploaded to an external network (e.g., a wireless computer network operated by a healthcare facility such as a hospital) via wireless transceiver 80 in base unit 10 (e.g., to be added to an Electronic Health Record (EHR)).

The electrical information acquired from the patient by novel apparatus 5 may be used directly by the healthcare professional to make a diagnosis and prognosis. Additionally and/or alternatively, the electrical information may be used by a computerized “comparison engine” or “auto-interpretation” engine (which can be incorporated in novel integrated, hand-held apparatus 5) to provide additional diagnostic and prognostic information to the healthcare professional.

Additionally, the electrical information acquired from the patient by novel apparatus 5 may be used by the healthcare professional to aid/guide the healthcare professional in therapeutic interventions.

Significantly, as noted above, novel integrated, hand-held apparatus 5 also provides the healthcare professional with access to patient records (e.g., an Electronic Health Record (EHR)) which may be available from an external network (e.g., a wireless computer network operated by a healthcare facility such as a hospital). Such access to patient records allows the current diagnostic and prognostic information acquired by novel integrated, hand-held apparatus 5 (e.g., a current ECG waveform) to be compared with the historic diagnostic and prognostic information in the patient records (e.g., an historic ECG waveform) which can assist the healthcare professional in the diagnosis of the patient. This comparison of current vs. historic diagnostic and prognostic information can be effected by the healthcare professional themselves or by a computerized “comparison engine” or “auto-interpretation engine” which can be incorporated in novel integrated, hand-held apparatus 5 or provided by the external network. Again, it will be appreciated that the “auto-interpretation engine” can compare the current diagnostic and prognostic information against a library of diagnostic and prognostic information and its anatomical meaning (e.g., heart murmur, valve issues, etc.) so as to provide further assistance to the heathcare provider.

### Blood Pressure Cuff

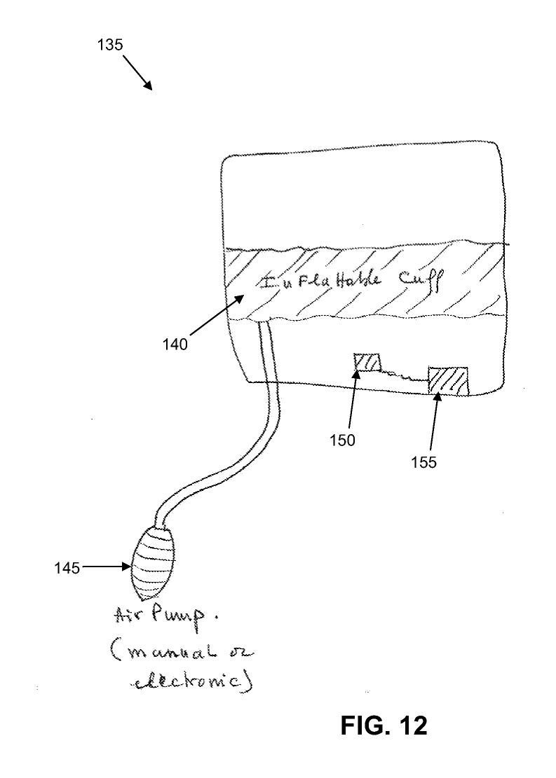

In still another preferred form of the present invention, and looking now at FIG. 12, novel integrated, hand-held apparatus 5 may also comprise a blood pressure cuff 135 for acquiring blood pressure information from the patient. To this end, blood pressure cuff 135 generally comprises an inflatable cuff 140, inflated by a manual or electrical air pump 145, for positioning a blood pressure sensor 150 against an artery of the patient. Blood pressure cuff 135 also comprises a wireless transceiver 155 (e.g., a Bluetooth device) for transmitting blood pressure information from blood pressure cuff 135 to base unit 10 (i.e., via the wireless transceiver 70 provided on base unit 10).

In accordance with the present invention, when the healthcare professional wishes to acquire blood pressure data from a patient at the bedside or at some other patient location, the healthcare professional positions blood pressure cuff 135 over an artery of the patient (e.g., around an arm of a patient), and then inflates inflatable cuff 140 using air pump 145. The blood pressure information acquired by blood pressure sensor 150 is then transmitted to base unit 10 via wireless transceiver 155 on blood pressure cuff 135 and wireless transceiver 70 on base unit 10. This blood pressure information may then be presented to the healthcare professional, e.g., visually via touchscreen display 55 and/or audibly via speaker 82. Preferably this blood pressure information is also simultaneously stored locally on base unit 10 in local data storage unit 65 and/or uploaded to an external network (e.g., a wireless computer network operated by a healthcare facility such as a hospital) via wireless transceiver 80 in base unit 10 (e.g., to be added to an Electronic Health Record (EHR)).

The blood pressure information acquired from the patient by novel apparatus 5 may be used directly by the healthcare professional to make a diagnosis and prognosis. Additionally and/or alternatively, the blood pressure information may be used by a computerized “comparison engine” or “auto-interpretation” engine (which can be incorporated in novel integrated, hand-held apparatus 5) to provide additional diagnostic and prognostic information to the healthcare professional.

Additionally, the blood pressure information acquired from the patient by novel apparatus 5 may be used by the healthcare professional to aid/guide the healthcare professional in therapeutic interventions.

In addition to the foregoing, as noted above, novel integrated, hand-held apparatus 5 may also acquire data (e.g., text, sounds, images, etc.) from an external network (e.g., a wireless computer network operated by a healthcare facility such as a hospital) via wireless transceiver 80 in base unit 10 (e.g., to access information from an Electronic Health Record (EHR)) and present that data to the healthcare professional via touchscreen display 55 and/or speaker 82. See FIG. 8. In this way, novel integrated, hand-held apparatus 5 also provides the healthcare professional with access to patient records which may be available from the external network, whereby to further assist the healthcare professional in the diagnosis of the patient. Significantly, such access to patient records allows the blood pressure information acquired by novel integrated, hand-held apparatus 5 to be compared with the historic diagnostic and prognostic information in the patient records, which can assist the healthcare professional in the diagnosis of the patient. This comparison of current vs. historic diagnostic and prognostic information can be effected by the healthcare professional themselves or by a computerized “comparison engine” or “auto-interpretation engine” which can be incorporated in novel integrated, hand-held apparatus 5 or provided by the external network. Again, it will be appreciated that the “auto-interpretation engine” can compare the current diagnostic and prognostic information against a library of diagnostic and prognostic information and its anatomical meaning (e.g., heart murmur, valve issues, etc.) so as to provide further assistance to the healthcare professional.

### Pulse Oximeter

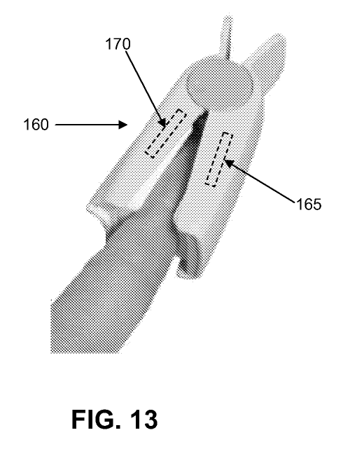

In still another preferred form of the present invention, and looking now at FIG. 13, novel integrated, hand-held apparatus 5 may also comprise a pulse oximeter 160 for acquiring pulse rate information and SpO₂ information from the body of the patient. To this end, pulse oximeter 160 comprises an appropriate pulse oximeter sensor 165 for positioning against the tissue of the patient (e.g., against a fingertip of a patient), and a wireless transceiver 170 (e.g., a Bluetooth device) for transmitting pulse oximeter information from pulse oximeter 160 to base unit 10 (i.e., via wireless transceiver 70 provided on base unit 10).

In accordance with the present invention, when the healthcare professional wishes to acquire pulse oximeter data from a patient at the bedside or at some other patient location, the healthcare professional positions pulse oximeter 160 against the tissue of the patient, i.e., so that pulse oximeter sensor 165 is positioned against the tissue of the patient. The pulse oximeter information acquired by pulse oximeter sensor 165 is then transmitted to base unit 10 via wireless transceiver 170 on pulse oximeter 160 and wireless transceiver 70 on base unit 10. This pulse oximeter information may then be presented to the healthcare professional, e.g., visually via touchscreen display 55 and/or audibly via speaker 82. Preferably this pulse oximeter information is also simultaneously stored locally on base unit 10 in local data storage unit 65 and/or uploaded to an external network (e.g., a wireless computer network operated by a healthcare facility such as a hospital) via wireless transceiver 80 in base unit 10 (e.g., to be added to an Electronic Health Record (EHR)).

The pulse rate information and SpO₂ information acquired from the patient by novel apparatus 5 may be used directly by the healthcare professional to make a diagnosis and prognosis. Additionally and/or alternatively, the pulse rate information and SpO₂ information may be used by a computerized “comparison engine” or “auto-interpretation” engine (which can be incorporated in novel integrated, hand-held apparatus 5) to provide additional diagnostic and prognostic information to the healthcare professional.

Additionally, the pulse rate information and SpO₂ information acquired from the patient by novel apparatus 5 may be used by the healthcare professional to aid/guide the healthcare professional in therapeutic interventions.

In addition to the foregoing, as noted above, novel integrated, hand-held apparatus 5 may also acquire data (e.g., text, sounds, images, etc.) from an external network (e.g., a wireless computer network operated by a healthcare facility such as a hospital) via wireless transceiver 80 in base unit 10 (e.g., to access information from an Electronic Health Record (EHR)) and present that data to the healthcare professional via touchscreen display 55 and/or speaker 82. See FIG. 8. In this way, novel integrated, hand-held apparatus 5 also provides the healthcare professional with access to patient records which may be available from the external network, whereby to further assist the healthcare professional in the diagnosis of the patient. Significantly, such access to patient records allows the blood oximeter information acquired by novel integrated, hand-held apparatus 5 to be compared with the historic diagnostic and prognostic information in the patient records, which can assist the healthcare professional in the diagnosis of the patient. This comparison of current vs. historic diagnostic and prognostic information can be effected by the healthcare professional themselves or by a computerized “comparison engine” or “auto-interpretation engine” which can be incorporated in novel integrated, hand-held apparatus 5 or provided by the external network. Again, it will be appreciated that the “auto-interpretation engine” can compare the current diagnostic and prognostic information against a library of diagnostic and prognostic information and its anatomical meaning (e.g., heart murmur, valve issues, etc.) so as to provide further assistance to the healthcare professional.

### Temperature Monitor

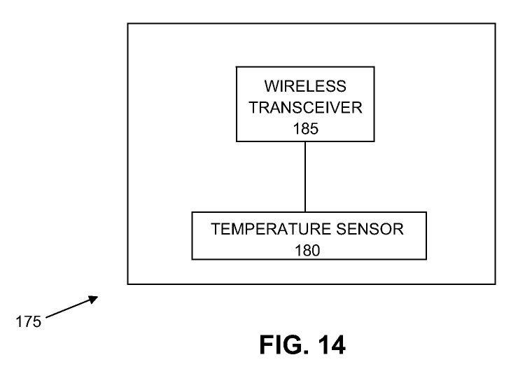

In still another preferred form of the present invention, and looking now at FIG. 14, novel integrated, hand-held apparatus 5 may also comprise a temperature monitor 175 for acquiring temperature information from the body of the patient. To this end, temperature monitor 175 comprises an appropriate temperature sensor 180 for positioning against the tissue of the patient, and a wireless transceiver 185 (e.g., a Bluetooth device) for transmitting temperature information acquired by temperature sensor 180 to base unit 10 (i.e., via wireless transceiver 70 provided on base unit 10).

In accordance with the present invention, when the healthcare professional wishes to acquire temperature data from a patient at the bedside or at some other patient location, the healthcare professional positions temperature monitor 175 against the tissue of the patient. The temperature information acquired by temperature sensor 180 is then transmitted to base unit 10 via wireless transceiver 185 on temperature monitor 175 and wireless transceiver 70 on base unit 10. This temperature information may then be presented to the healthcare professional, e.g., visually via touchscreen display 55 and/or audibly via speaker 82. Preferably this temperature information is also simultaneously stored locally on base unit 10 in local data storage unit 65 and/or uploaded to an external network (e.g., a wireless computer network operated by a healthcare facility such as a hospital) via wireless transceiver 80 in base unit 10 (e.g., to be added to an Electronic Health Record (EHR)).

The temperature information acquired from the patient by novel apparatus 5 may be used directly by the healthcare professional to make a diagnosis and prognosis. Additionally and/or alternatively, the temperature information may be used by a computerized “comparison engine” or “auto-interpretation” engine (which can be incorporated in novel integrated, hand-held apparatus 5) to provide additional diagnostic and prognostic information to the healthcare professional.

Additionally, the temperature information acquired from the patient by novel apparatus 5 may be used by the healthcare professional to aid/guide the healthcare professional in therapeutic interventions.

In addition to the foregoing, as noted above, novel integrated, hand-held apparatus 5 may also acquire data (e.g., text, sounds, images, etc.) from an external network (e.g., a wireless computer network operated by a healthcare facility such as a hospital) via wireless transceiver 80 in base unit 10 (e.g., to access information from an Electronic Health Record (EHR)) and present that data to the healthcare professional via touchscreen display 55 and/or speaker 82. See FIG. 8. In this way, novel integrated, hand-held apparatus 5 also provides the healthcare professional with access to patient records which may be available from the external network, whereby to further assist the healthcare professional in the diagnosis of the patient. Significantly, such access to patient records allows the temperature information acquired by novel integrated, hand-held apparatus 5 to be compared with the historic diagnostic and prognostic information in the patient records, which can assist the healthcare professional in the diagnosis of the patient. This comparison of current vs. historic diagnostic and prognostic information can be effected by the healthcare professional themselves or by a computerized “comparison engine” or “auto-interpretation engine” which can be incorporated in novel integrated, hand-held apparatus 5 or provided by the external network. Again, it will be appreciated that the “auto-interpretation engine” can compare the current diagnostic and prognostic information against a library of diagnostic and prognostic information and its anatomical meaning (e.g., heart murmur, valve issues, etc.) so as to provide further assistance to the healthcare professional.

### Additional Features

If desired, features in addition to those disclosed above may be included in novel integrated, hand-held apparatus 5 so as to provide the device with additional functionality.

By way of example but not limitation, novel apparatus 5 may also be provided with a voice microphone 190 (FIGS. 2 and 3) for audio recording, e.g., to record the voice of the healthcare professional, the voice of the patient or some other sound of the patient (e.g., a cough), or the voice of some other individual. The voice data acquired by voice microphone 190 is preferably stored locally on base unit 10 in local data storage unit 65 and/or uploaded to an external data network (e.g., a wireless computer network operated by a healthcare facility such as a hospital) via wireless transceiver 80 in base unit 10 (e.g., to be added to an Electronic Health Record (EHR)). In one preferred form of the invention, voice microphone 190 may be disposed on base unit 10 (FIGS. 2 and 3). In another preferred form of the invention, voice microphone 190 may be disposed on wand 15, and the information acquired by voice microphone 190 may be relayed to base unit 10 via wireless transceiver 105 in wand 15 and wireless transceiver 70 in base unit 10. If voice microphone 190 is disposed on wand 15, voice microphone 190 may be combined with the microphone 95 previously discussed.

By way of further example but not limitation, novel integrated, hand-held apparatus 5 may also be provided with a camera 195 for taking pictures, e.g., to visually record the appearance of patient anatomy. Preferably camera 195 is also provided with appropriate functionality of the sort well known in the art to permit barcode scanning by means of camera 195, e.g., so as to read the barcode on a patient's bracelet. The image data acquired by camera 195 is preferably stored locally on base unit 10 in local data storage unit 65 and/or uploaded to an external data network (e.g., a wireless computer network operated by a healthcare facility such as a hospital) via wireless transceiver 80 in base unit 10 (e.g., to be added to an Electronic Health Record (EHR)).

By way of further example but not limitation, speaker 82 on base unit 10 may be supplemented by providing an earphone jack 200 (FIGS. 2 and 3) for receiving the input of an earphone. Earphone jack 200 can be very helpful for the healthcare professional where there is a substantial amount of ambient noise about the patient, or where there is a need for quiet about the patient. In this respect it should also be appreciated that earphone jack 200 and/or its associated earphone may replaced by a corresponding headphone jack/headphone arrangement which provides a microphone for dictation by the healthcare professional as well as earphones for delivering sound information to the healthcare professional. In this situation, dictation by the healthcare professional is preferably stored locally on base unit 10 in local data storage unit 65 and/or uploaded to an external data network (e.g., a wireless computer network operated by a healthcare facility such as a hospital) via wireless transceiver 80 in base unit 10 (e.g., to be added to an Electronic Health Record (EHR)).

By way of still further example but not limitation, base unit 10 of novel integrated, hand-held apparatus 5 may incorporate appropriate programming so as to provide the healthcare professional with an Internet browser, with base unit 10 accessing the Internet via the wireless transceiver 80 which communicates with an external network (e.g., a wireless computer network operated by a healthcare facility such as a hospital). This arrangement can provide the healthcare professional with access to information and services available on the Internet, e.g., publications, E-mail, telephone, text-paging, text messages (including sms), etc.

By way of still further example but not limitation, base unit 10 may be configured to provide additional helpful information to the healthcare professional so as to improve patient care and/or healthcare professional efficiency, e.g., base unit 10 may provide the healthcare professional with a list of the patients who are to be seen by that healthcare professional, etc.

### Wired vs. Wireless Communications

If desired some or all of the wireless connections associated with novel integrated, hand-held apparatus 5 may be replaced by a hard-wired link. Thus, the wireless connection between base unit 10 and wand 15 (i.e., the wireless transceiver 70 in base unit 10 and the wireless transceiver 105 in wand 15) may be replaced by a hard-wired link; and/or the wireless connection between base unit 10 and an external network (i.e., the wireless transceiver 80 in base unit 10 and a corresponding wireless transceiver in the external network) may be replaced by a hard-wired link; and/or the wireless connection between base unit 10 and blood pressure cuff 135 (i.e., the wireless transceiver 70 in base unit 10 and the wireless transceiver 155 in blood pressure cuff 135) may be replaced by a hard-wired link; and/or the wireless connection between base unit 10 and pulse oximeter 160 (i.e., the wireless transceiver 70 in base unit 10 and the wireless transceiver 170 in blood oximeter 160) may be replaced by a hard-wired link; and/or the wireless connection between base unit 10 and temperature monitor 175 (i.e., the wireless transceiver 70 in base unit 10 and the wireless transceiver 185 in temperature monitor 175) may be replaced by a hard-wired link. Furthermore, voice microphone 190 may be replaced by a wireless microphone, and/or earphone jack 200 (and the associated earphone) may be replaced by a wireless earphone, and/or the headphone jack/headphone arrangement may be replaced by a wireless headphone, etc.

### Docking Station

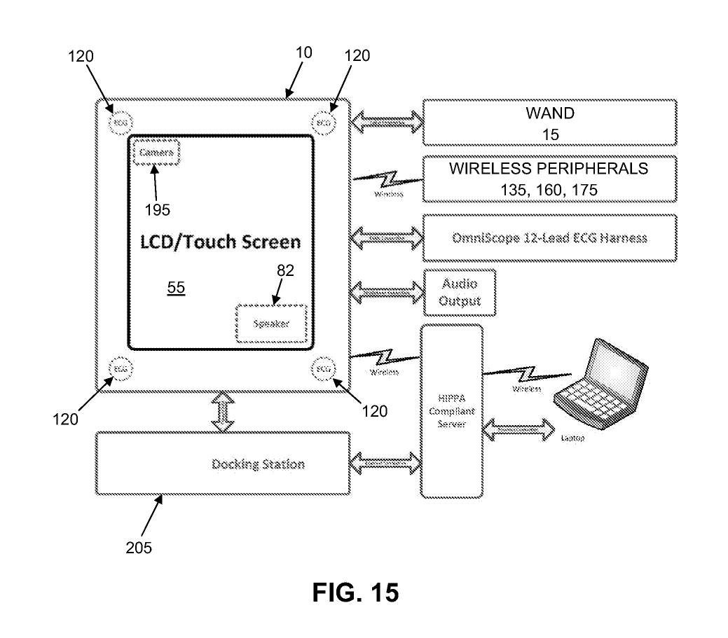

If desired, and looking now at FIG. 15, a docking station 205 may be provided for base unit 10 of novel integrated, hand-held apparatus 5. This docking station can provide battery recharging functions, as well as data download and upload functions, for base unit 10.

### Apparatus Architecture

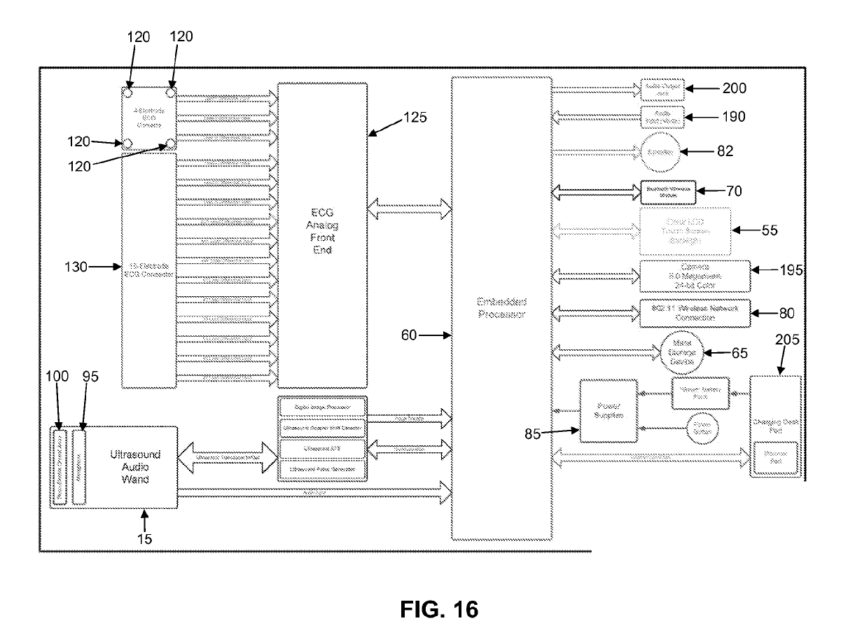

It will be appreciated that various architectures may be employed in the construction of novel integrated, hand-held apparatus 5. FIG. 16 shows one exemplary system block diagram for novel integrated, hand-held apparatus 5. Other system architectures will be apparent to those skilled in the art in view of the present disclosure.

### Additional Wand Configurations

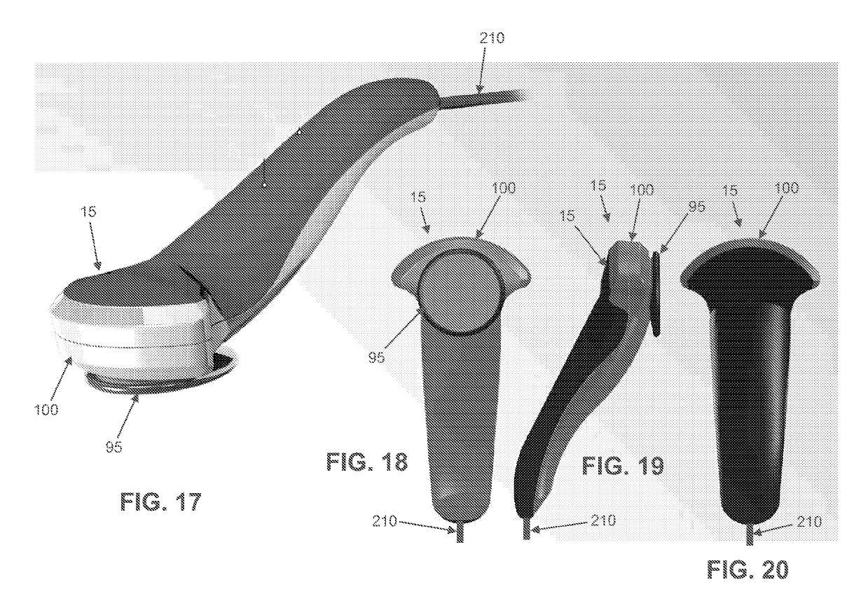

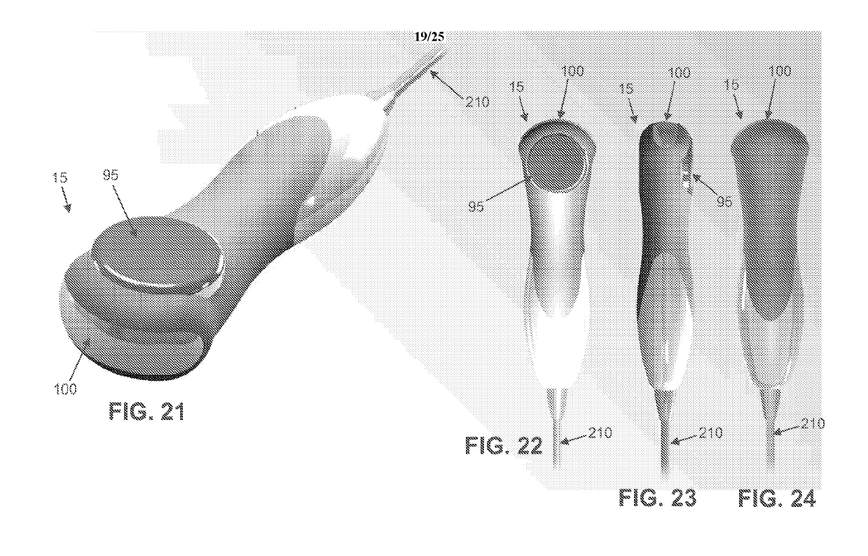

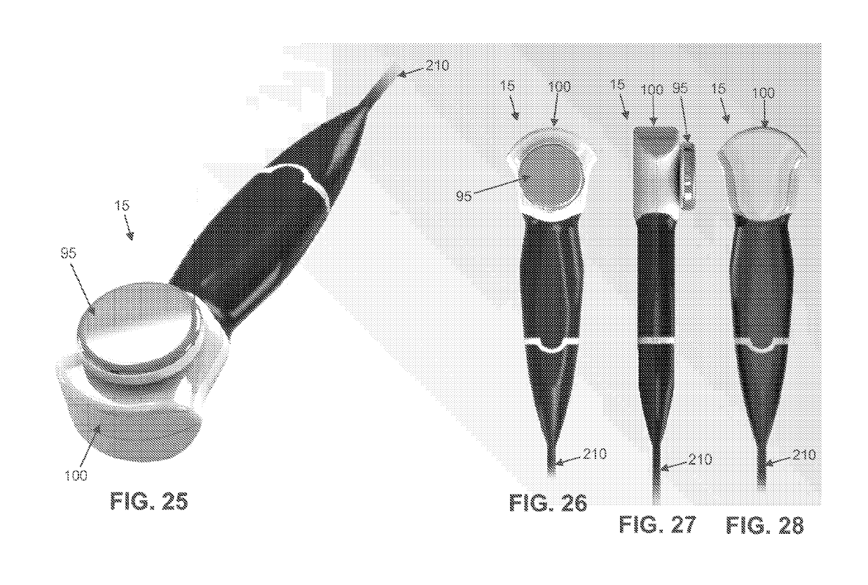

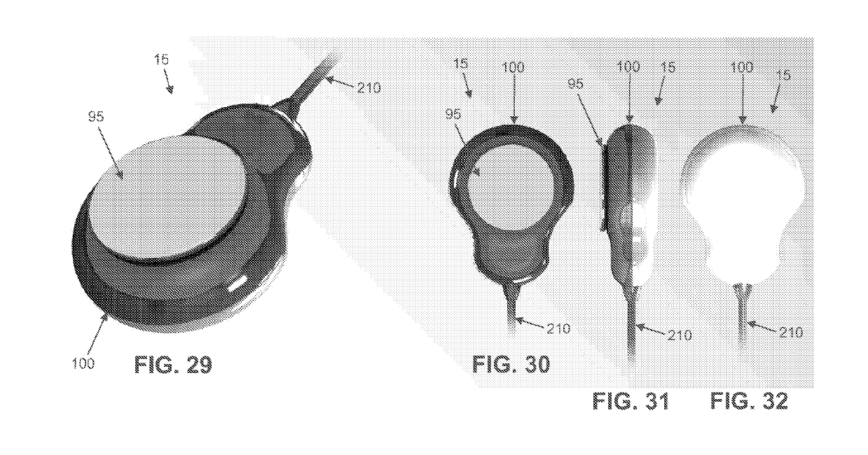

As noted above, wand 15 may be formed with an elongated, generally cylindrical configuration (FIGS. 1, 2 and 4-7) or a more complex configuration (FIGS. 9 and 10). FIGS. 17-32 show still other possible configurations for wand 15. Furthermore, the wands 15 shown in FIGS. 17-32 are provided with a wire 210 for connecting wand 15 to base unit 10 via a hard-wired connection.

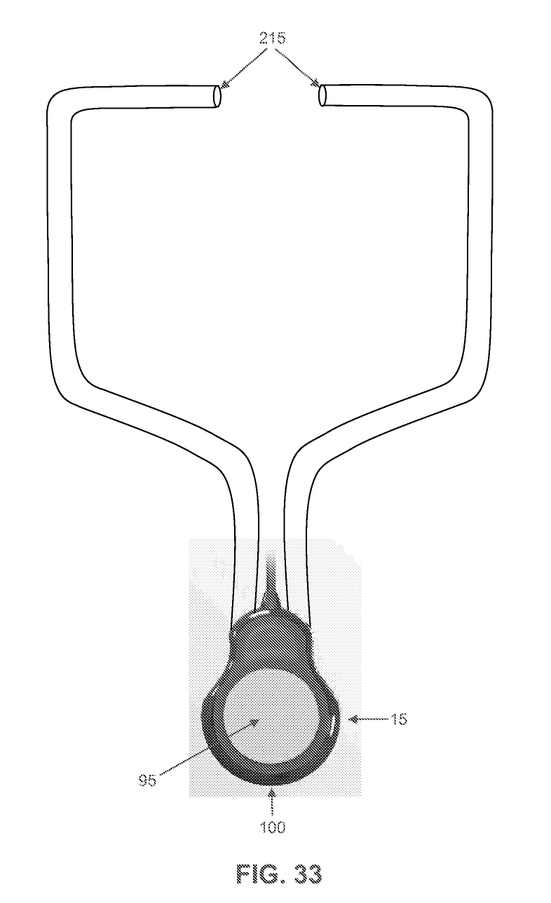

Additionally, if desired, wand 15 can be provided with earpieces 215 (see FIG. 33), whereby to provide an ergonomic construction (the wand and earpieces are formed as a single unit) having a familiar feel and appearance to the healthcare professional and the patient (the healthcare professional manipulates the wand in a manner somewhat analogous to the head of a conventional stethoscope).

### “Unibody” Construction

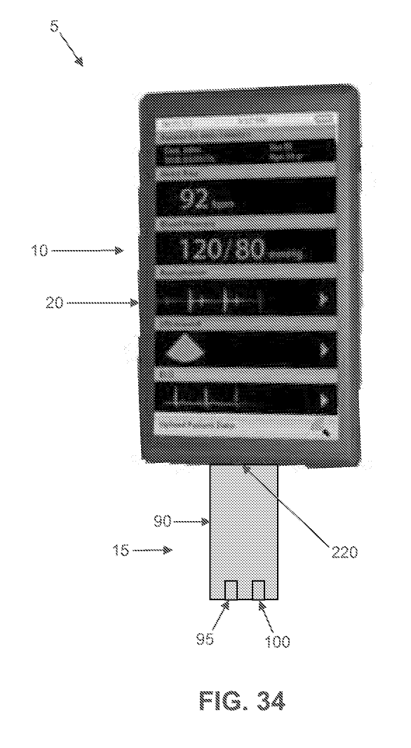

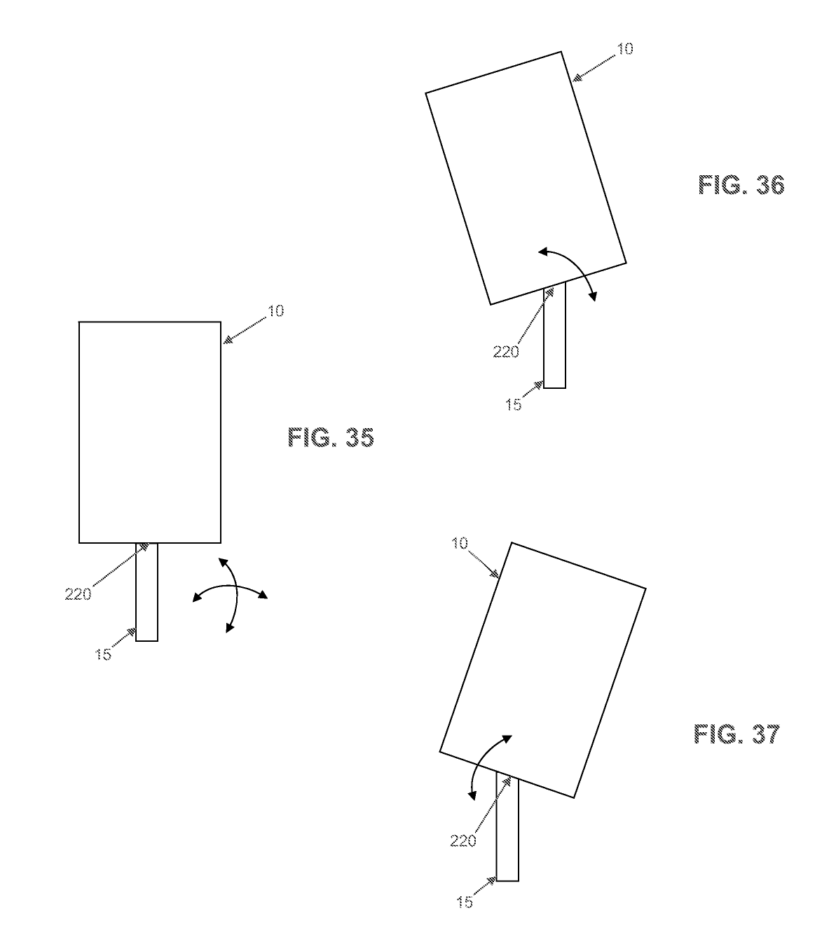

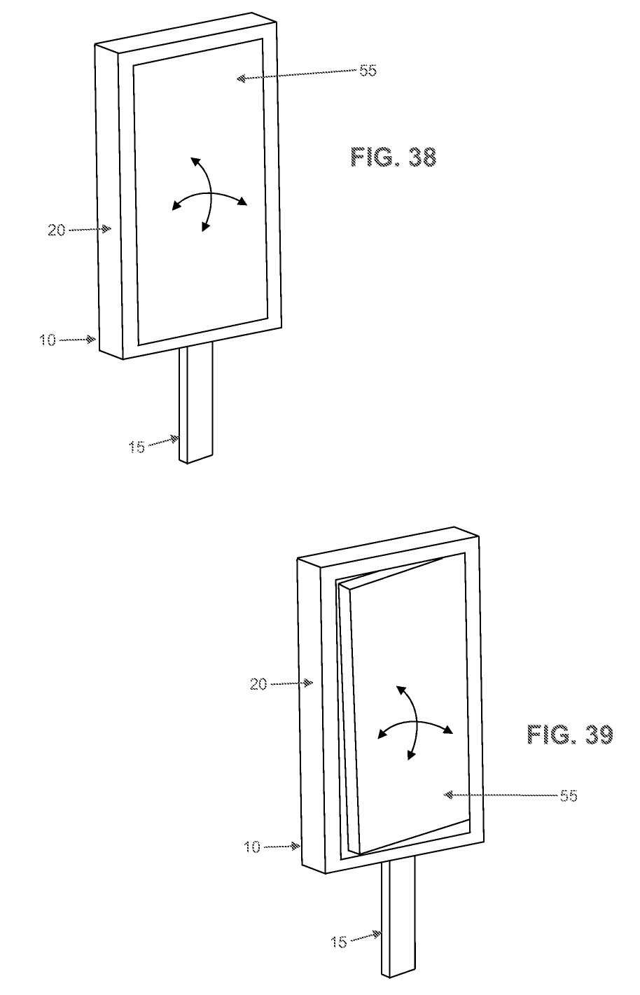

It should also be appreciated that, if desired, base unit 10 and wand 15 may be combined together in a “unibody” construction, such that body 90 of wand 15 is mounted to, and extends from, body 20 of base unit 10 and, if desired, can be retracted into base unit 10. See FIG. 34. In this form of the invention, apparatus 5 may be provided with a multi-directional hinge 220 (or a gooseneck mechanism, etc.) at the intersection of wand 15 and base unit 10 so that touchscreen display 55 can be appropriately angled for easy reading by the heathcare professional when wand 15 is appropriately contacting the patient. See FIGS. 35-37. Alternatively, touchscreen display 55 may be mounted to body 20 of base unit 10 by an internal pivoting hinge mechanism (not shown) so that touchscreen display 55 can be appropriately angled for easy reading by the healthcare professional when wand 15 is appropriately contacting the patient. See FIGS. 38 and 39.

### Example

In one exemplary construction:

the audio transducer of wand 15 is capable of measuring input signal amplitudes of 20 dB minimum, the frequency response of the audio transducer has an accuracy of +/−2 dB in the 50 Hz to 10 kHz range, the auscultation circuitry has a dynamic range of 40 dB minimum, the auscultation circuitry has an SNR of 40 dB minimum and the sampling frequency of the audio input signal is no less than 48 kSPS;

the ultrasound transducer of wand 15 produces ultrasonic energy waves in the range of 2 MHz to 7 MHz, the ultrasound system is capable of detecting echos from depths of 1 cm to 20 cm from the face of the wand, the ultrasound system is capable of steering the focused energy in a plane of up to 90 degrees centered around the parallel axis of the wand, the ultrasound system includes a band-pass filter with corner frequencies of 2 and 7 MHz, the ultrasound system digitizes each ultrasound reading with a resolution of no less than 10 bits, the ultrasound system has a spatial resolution of no more than 1 mm, the ultrasound system has a temporal resolution of no more than 300 μs per transmitted pulse, the ultrasound system digitizes the ultrasound reading with a sampling frequency of no less than 20 MSPS, the ultrasound system is capable of processing and displaying B-mode ultrasound images, the ultrasound system is capable of processing and displaying color Doppler ultrasound images, the ultrasound system is capable of processing and displaying pulsed-wave Doppler ultrasound images, the ultrasound system includes 256 shades of gray in the output amplitude image, the ultrasound system includes 256 shades of color in the color/pulsed-wave Doppler images, the color/pulsed-wave Doppler image shading displays a red-yellow color at a step value of −128 corresponding to a positive frequency shift, the color/pulsed-wave Doppler image shading displays a blue-green color at a step value of 128 corresponding to a negative frequency shift, the color/pulsed-wave Doppler images display black when no Doppler shift has occurred (step value of 0), the ultrasound image is updated at a minimum rate of 30 Hz for B-mode imaging and 15 Hz for color/pulsed-wave Doppler image;

the touchscreen display is an LCD device with a contrast ratio of no less than 5:1 and has a brightness rating of no less than 700 nits;

the ECG inputs have an input impedance of no less than 10 MΩ when a DC voltage is applied, the ECG inputs have ESD protection for voltage spikes of up to 10 kV, the ECG valid signal input range is a minimum of +/−3 mVAC, the ECG system has a CMRR of 60 dB minimum at 60 Hz and 45 dB minimum at 120 Hz, the ECG system meets all requirements with up to +/−300 mVDC applied to the electrodes, the ECG system includes a band-pass filter with corner frequencies of 0.1 Hz and 100 Hz, the signal gain of the ECG system in the range of 0.5 Hz to 30 Hz does not vary by more than +/−15% of the gain at 5 Hz, the ECG system digitizes the input signals at a sampling rate of no less than 200 SPS;

the blood pressure cuff is capable of pressurizing the cuff bladder up to 300 mm Hg;

the pulse oximeter reports the SpO₂ as a percentage with one decimal place, the pulse oximeter reports SpO₂ with an accuracy of +/−0.1% SpO₂;

the voice microphone is capable of measuring sound amplitudes of up to 70 dB, the voice microphone is capable of measuring sound amplitudes down to 20 dB with an SNR of 40 dB minimum, the voice microphone includes a band-pass filter with corner frequencies of 20 Hz and 20 kHz, the system digitizes the input of the voice microphone at a sampling frequency of no less than 48 kSPS;

the system is capable of outputting up to 70 dB of audio sound from 100 Hz to 10 kHz measured at 12 inches from the front face of the system unit; and

the camera has image resolutions of up to 5 megapixels, the camera is capable of focusing an object in the range of 0.5 feet to 3 feet from the camera face, and the camera is ready to capture a new image within 5 seconds of capturing a previous image.

### Advantages of the Present Invention

Thus it will be seen that novel apparatus 5 comprises an integrated, hand-held device which is intended to be conveniently carried by a healthcare professional on their person (e.g., in the manner of a conventional stethoscope) and which can be used to acquire sound, image and preferably also electrical and other (e.g., patient history, blood pressure, blood oximetry, patient temperature, etc.) information from the patient, so as to enable the healthcare professional to carry out a rapid, accurate and comprehensive objective physical examination of the patient at the bedside or at some other patient location, including cardiovascular diagnostics and prognostics, regardless of any other equipment that may be available at that location, and/or to aid/guide the healthcare professional in therapeutic interventions (e.g., localization of pericardial or pleural effusions and guiding a needle/catheter to drain the fluid), and to store the diagnostic and prognostic information acquired from the patient, either locally on the device or externally on an external network, for later review by that same healthcare professional and/or by others. Significantly, because the novel integrated, hand-held apparatus of the present invention facilitates the acquisition of objective diagnostic and prognostic information from the patient, the present invention facilitates a more accurate and prompt diagnosis and prognosis of patient conditions by the clinician and also allows a broader set of healthcare professionals (e.g., technicians and others who will not actually render a diagnosis) to be involved in the acquisition of diagnostic and prognostic information from the patient.

By way of example but not limitation, having sound information (auscultation) and image data (ultrasound) simultaneously available to the healthcare professional at the bedside or some other patient location may be extremely useful in properly diagnosing and identifying fluid build-ups in the body (e.g., in the chest, limbs and abdomen) such as by palpation in conjunction with sound information and/or image data, determining appropriate biopsy sites and obtaining desired biopsy specimens, identifying and accessing (e.g., via instrument guidance) desired interventional sites, accurately locating blood vessels, etc. Furthermore, the use of sound information (auscultation) with Doppler ultrasound could lead to better specificity and better diagnosis of a patient condition than sound information (auscultation) alone (e.g., immediate diagnosis of murmurs or bruits).

Furthermore, having two or more diagnostic and prognostic functions (e.g., sound information and image data) simultaneously available to the healthcare professional at the bedside or some other patient location could provide additional significant advantages. By way of example but not limitation, the following advantages could be obtained:
(i) accuracy advantage—by having two or more functions immediately at hand (instead of available as the result of performing two or more tests separated over time), the healthcare professional could be better able to localize a problem (e.g., the healthcare professional could hear an issue via auscultation, and could then immediately run an ultrasound to visualize the problem, instead of having to wait for the results of a separate ultrasound test performed by a different healthcare professional at a later time);
(ii) diagnostic advantage—by having two or more functions immediately at hand (instead of available as the result of performing two or more tests separated over time), the healthcare professional could be better able to isolate/detect a problem while the patient is actually experiencing the problem (e.g., the healthcare professional could hear, see and correlate the problem with other key biological measures while the patient is experiencing and describing the symptoms, instead of having to wait for the results of later-scheduled tests);
(iii) prognostic advantage—by having two or more functions immediately at hand (instead of available as the result of performing two or more tests separated over time), the healthcare professional could be better able to gauge the severity of a problem (e.g., the healthcare professional could hear, see and correlate the problem with other key biological measures contemporaneously, as opposed to having to wait for the results of tests separated over time);
(iv) economic advantage—by having two or more functions immediately at hand (instead of available as the result of performing two or more tests separated over time), it may be possible to avoid multiple patient trips to a healthcare facility and to avoid involving multiple healthcare professionals in the process, thereby reducing patient inconvenience and patient opportunity cost, reducing administrative costs, and increasing the productivity of the healthcare professionals;
(v) treatment advantage—by having two or more functions immediately at hand (instead of available as the result of performing two or more tests separated over time), there could be a reduction of the time between tests, which could permit treatment to be more rapidly administered to the patient and could potentially avoid a worsening of the patient's condition;
(vi) convenience advantage—by having two or more functions immediately at hand (instead of available as the result of two or more tests separated over time), there could be a reduction in the number of appointments which need to be scheduled by/for the patient and there could be a reduction in the number of immediate follow-up appointments which need to be scheduled by/for the patient;
(vii) health advantage—by having two or more functions immediately at hand (instead of available as the result of two or more tests separated over time), there could be a reduction of the time during which the patient may be anxious about an undiagnosed condition, thereby reducing overall patient anxiety and potentially improving patient health as a result (e.g., there could be a reduction of the time during which the patient is waiting for an appointment for a test to be conducted, for the results to be acquired by a technician, for the results to be provided by the technician to the doctor, for the results to be interpreted by the doctor, and for the results to be provided by the doctor to the patient, during which time the patient may be suffering through the anxiety of having an undiagnosed condition).

Still other advantages will be apparent to those skilled in the art in view of the present disclosure.

### Modifications of the Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. An integrated, hand-held apparatus for acquiring diagnostic and prognostic information from a patient at the bedside or at some other patient location, the apparatus comprising:
a wand comprising:
a microphone for acquiring sound information from the patient; and
an ultrasound emitter/receiver for acquiring image data from the patient; and
a base unit comprising:
a speaker for presenting sound information to a user; and
a display for presenting image information to a user; and
transferring means for transferring the sound information acquired by the microphone, and the image information acquired by the ultrasound emitter/receiver, from the wand to the base unit.

2. Apparatus according to claim 1 wherein the wand is releasably secured to the base unit.

3. Apparatus according to claim 1 wherein the base unit is configured to present sound information as image information on the display.

4. Apparatus according to claim 1 wherein the transferring means are configured to wirelessly transfer the sound information acquired by the microphone, and the image information acquired by the ultrasound emitter/receiver, from the wand to the base unit.

5. Apparatus according to claim 4 wherein the base unit comprises a base unit wireless transceiver, the wand comprises a wand wireless transceiver, and the wand wireless transceiver is configured to communicate with the base unit wireless transceiver.

6. Apparatus according to claim 5 wherein the base unit wireless transceiver and the wand wireless transceiver are Bluetooth devices.

7. Apparatus according to claim 1 wherein the transferring means are configured to transfer the sound information acquired by the microphone, and the image information acquired by the ultrasound emitter/receiver, from the wand to the base unit via a hardwire connection.

8. Apparatus according to claim 1 wherein the base unit further comprises a local data storage unit for locally storing sound information and image information.

9. Apparatus according to claim 1 wherein the base unit further comprises communication means for communicating with an external network.

10. Apparatus according to claim 9 wherein the communication means is configured to send the sound information acquired by the microphone, and the image information acquired by the ultrasound emitter/receiver, to the external network.

11. Apparatus according to claim 9 wherein the communication means is configured to acquire information from the external network and present it to a user using at least one of the speaker and the display.

12. Apparatus according to claim 9 wherein the communication means comprises a wireless transceiver.

13. Apparatus according to claim 12 wherein the wireless transceiver is a WiFi device.

14. Apparatus according to claim 1 further comprising comparison means for comparing the sound information acquired by the microphone with a database of sound information.

15. Apparatus according to claim 1 further comprising comparison means for comparing the image information acquired by the ultrasound emitter/receiver with a database of image information.

16. Apparatus according to claim 1 wherein the base unit comprises a plurality of electrodes for acquiring electrical information from the patient, and means for displaying the electrical information acquired from the patient on at least one of the display and the speaker.

17. Apparatus according to claim 1 wherein the base unit comprises a connector for connecting a standard 12-lead ECG electrode array to the base unit, and means for displaying the electrical information acquired via the standard 12-lead ECG electrode array on at least one of the display and the speaker.

18. Apparatus according to claim 16 further comprising comparison means for comparing the electrical information acquired by the plurality of electrodes with a database of electrical information.

19. Apparatus according to claim 1 further comprising a blood pressure cuff for acquiring blood pressure information from the patient, means for transferring the blood pressure information from the blood pressure cuff to the base unit, and means for displaying blood pressure information on at least one of the display and the speaker.

20. Apparatus according to claim 19 further comprising comparison means for comparing the blood pressure acquired by the blood pressure cuff with a database of blood pressure information.

21. Apparatus according to claim 1 further comprising a pulse oximeter for acquiring pulse rate and SpO₂ information from the patient, means for transferring the pulse rate and SpO₂ information from the pulse oximeter to the base unit, and means for displaying pulse rate and SpO₂ information on at least one of the display and the speaker.

22. Apparatus according to claim 21 further comprising comparison means for comparing the pulse rate and SpO₂ information acquired by the pulse oximeter with a database of pulse rate and SpO₂ information.

23. Apparatus according to claim 1 further comprising a temperature sensor for acquiring temperature information from the patient, means for transferring the temperature information from the temperature sensor to the base unit, and means for displaying temperature information on at least one of the display and the speaker.

24. Apparatus according to claim 23 further comprising comparison means for comparing the temperature information acquired by the temperature sensor with a database of temperature information.

25. Apparatus according to claim 1 wherein the base unit comprises a camera for acquiring visual information from a patient, and means for displaying visual information on the display.

26. Apparatus according to claim 1 wherein the wand comprises an elongated body, and further wherein the microphone and the ultrasound emitter/receiver are both presented at an end of the body.

27. Apparatus according to claim 26 wherein the microphone and the ultrasound emitter/receiver are presented at the same end of the body.

28. Apparatus according to claim 26 wherein the microphone and the ultrasound emitter/receiver are disposed adjacent to one another.

29. Apparatus according to claim 28 wherein the microphone and the ultrasound emitter/receiver are disposed longitudinally adjacent to one another.

30. Apparatus according to claim 28 wherein the microphone and the ultrasound emitter/receiver are disposed laterally adjacent to one another.

31. Apparatus according to claim 26 wherein the microphone is presented at one end of the body and the ultrasound emitter/receiver is presented on an opposing end of the body.

32. Apparatus according to claim 26 wherein the microphone is presented intermediate the body and the ultrasound emitter/receiver is presented at one end of the body.

33. Apparatus according to claim 1 wherein the microphone and the ultrasound emitter/receiver share at least one common element.

34. Apparatus according to claim 1 wherein the wand and the base unit have a unibody construction.

35. Apparatus according to claim 34 wherein the wand is movably hinged to the base unit.

36. Apparatus according to claim 34 wherein the display is movably hinged to the base unit.

37. A wand comprising:
a microphone for acquiring sound information from the patient; and
an ultrasound emitter/receiver for acquiring image data from the patient.

38. An integrated, hand-held apparatus for acquiring diagnostic and prognostic information from a patient at the bedside or at some other patient location, the apparatus comprising:
a wand comprising:
a microphone for acquiring sound information from the patient; and
an ultrasound emitter/receiver for acquiring image data from the patient;
a base unit comprising:
a speaker for presenting sound information to a user; and
a display for presenting image information to a user;
transferring means for transferring the sound information acquired by the microphone, and the image information acquired by the ultrasound emitter/receiver, from the wand to the base unit;
a plurality of electrodes for acquiring electrical information from the patient, and means for displaying the electrical information acquired from the patient on at least one of the display and the speaker;
a blood pressure cuff for acquiring blood pressure information from the patient, means for transferring the blood pressure information from the blood pressure cuff to the base unit, and means for displaying blood pressure information on at least one of the display and the speaker;
a pulse oximeter for acquiring pulse rate and SpO₂ information from the patient, means for transferring the pulse rate and SpO₂ information from the blood pulse oximeter to the base unit, and means for displaying pulse rate and SpO₂ information on at least one of the display and the speaker;
a temperature sensor for acquiring temperature information from the patient, means for transferring the temperature information from the temperature sensor to the base unit, and means for displaying temperature information on at least one of the display and the speaker; and
communication means for permitting the apparatus to communicate with an external network.

39. A method for acquiring diagnostic and prognostic information from a patient at the bedside or at some other patient location, the method comprising:
providing an integrated, hand-held apparatus comprising:
a wand comprising:
a microphone for acquiring sound information from the patient; and
an ultrasound emitter/receiver for acquiring image data from the patient; and
a base unit comprising:
a speaker for presenting sound information to a user; and
a display for presenting image information to a user; and
transferring means for transferring the sound information acquired by the microphone, and the image information acquired by the ultrasound emitter/receiver, from the wand to the base unit; and
acquiring at least one of sound information and image information from the patient using the apparatus.

40. A method according to claim 39 further comprising presenting at least one of sound information and image information to the user on at least one of the speaker and the display.

41. A method according to claim 40 wherein the diagnostic and prognostic information is sound information, and further wherein the sound information is presented as image information on the display.

42. A method according to claim 39 wherein the transferring means are configured to wirelessly transfer the sound information acquired by the microphone, and the image information acquired by the ultrasound emitter/receiver, from the wand to the base unit.

43. A method according to claim 39 wherein the transferring means are configured to transfer the sound information acquired by the microphone, and the image information acquired by the ultrasound emitter/receiver, from the wand to the base unit via a hardwire connection.

44. A method according to claim 39 further comprising storing at least one of sound information and image information on the base unit in a local data storage unit.

45. A method according to claim 39 wherein the base unit further comprises communication means for communicating with an external network.

46. A method according to claim 45 further comprising sending at least one of the sound information acquired by the microphone, and/or the image information acquired by the ultrasound emitter/receiver, to the external network via the communication means.

47. A method according to claim 45 wherein the communication means is configured to acquire information from the external network and present it to a user using at least one of the speaker and the display.

48. A method according to claim 45 wherein the communication means comprises a wireless transceiver.

49. A method according to claim 39 further comprising comparing the sound information acquired by the microphone with a database of sound information.

50. A method according to claim 39 further comprising comparing the image information acquired by the ultrasound emitter/receiver with a database of image information.

51. A method according to claim 39 wherein the base unit comprises a plurality of electrodes for acquiring electrical information from the patient, and means for displaying the electrical information acquired from the patient on at least one of the display and the speaker, and further wherein the method comprises displaying the electrical information acquired from the patient on at least one of the display and the speaker.

52. A method according to claim 39 wherein the base unit comprises a connector for connecting a standard 12-lead ECG electrode array to the base unit, and means for displaying the electrical information acquired via the standard 12-lead ECG electrode array on at least one of the display and the speaker, and further wherein the method comprises displaying the electrical information acquired from the patient on at least one of the display and the speaker.

53. A method according to claim 51 further comprising comparing the electrical information acquired by the plurality of electrodes with a database of electrical information.

54. A method according to claim 39 further comprising a blood pressure cuff for acquiring blood pressure information from the patient, means for transferring the blood pressure information from the blood pressure cuff to the base unit, and means for displaying blood pressure information on at least one of the display and the speaker, and further wherein the method comprises displaying the blood pressure information acquired from the patient on at least one of the display and the speaker.

55. A method according to claim 54 further comprising comparing the blood pressure acquired by the blood pressure cuff with a database of blood pressure information.

56. A method according to claim 39 further comprising a pulse oximeter for acquiring pulse rate and SpO₂ information from the patient, means for transferring the pulse rate and SpO₂ information from the blood pulse oximeter to the base unit, and means for displaying pulse rate and SpO₂ information on at least one of the display and the speaker, and further wherein the method comprises displaying the pulse rate and the SpO₂ information acquired from the patient on at least one of the display and the speaker.

57. A method according to claim 56 further comprising comparing the pulse rate and SpO₂ information acquired by the pulse oximeter with a database of pulse rate and SpO₂ information.

58. A method according to claim 39 further comprising a temperature sensor for acquiring temperature information from the patient, means for transferring the temperature information from the temperature sensor to the base unit, and means for displaying temperature information on at least one of the display and the speaker, and further wherein the method comprises displaying the temperature information acquired from the patient on at least one of the display and the speaker.

59. A method according to claim 58 further comprising comparing the temperature information acquired by the temperature sensor with a database of temperature information.

60. A method according to claim 39 wherein the base unit comprises a camera for acquiring visual information from a patient, and means for displaying visual information on the display, and further wherein the method comprises displaying the visual information acquired from the patient on the display.

61. A method according to claim 39 further comprising performing a therapeutic intervention using at least one of the acquired sound information and the acquired image information.

62. A method according to claim 39 further comprising performing a therapeutic intervention using both the acquired sound information and the acquired image information.
